(19) United States
(12) Patent Application Publication
(10) Pub. No.: US 2006/0251688 A1
(43) Pub. Date: Nov. 9, 2006

(54) Polyurethane microcapsules containing biocide and process for the preparation thereof

(75) Inventors: Shukla Parshuram Gajanan, Pune (IN); Swaminathan Sivaram, Pune (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH.

(21) Appl. No.: 11/355,253

(22) Filed: Feb. 15, 2006

(30) Foreign Application Priority Data
Feb. 28, 2005 (IN) 427/DEL/2005

(51) Int. Cl. A61K 8/02; A61K 9/14; B01J 13/04

(52) U.S. Cl. 424/401; 424/489; 264/4.1

(57) ABSTRACT

The invention provides polyurethane microcapsules containing biocide, useful for preparing coating materials such as interior and exterior paints. Microcapsules described in this invention are prepared by dispersing biocide in an aliphatic hydrocarbon medium containing nonionic polymeric stabilizer having hydrophilic and hydrophobic repeating units, diol or polyol having molecular weight 200-2000, crosslinker and a catalyst selected from amino or organometallic compounds; adding an isocyanate drop wise to this dispersion; agitating the mixture at 800-1000 rotations per minute for the first 3-5 hours at 40-50° C. and then at 12-15 hours at 20-27° C.; to permit the formation of polyurethane microcapsules; filtering and washing the microcapsules with lower aliphatic hydrocarbon and drying the microcapsules under vacuum at temperature between 20-35° C.

### FIELD OF THE INVENTION

This invention relates to polyurethane microcapsules containing biocide and process for the preparation thereof. More particularly, the invention describes preparation of polyurethane microcapsules containing biocide, especially Irgarol (algaecide) and Zinc Pyrithione (fungicide). Microcapsules obtained by the process of the present invention range in size from one micron to hundred microns.

### BACKGROUND OF THE INVENTION

Biocides are chemical compounds, which are toxic to microbial cells and are added to different types of products to prevent the growth of unwanted microorganisms. Reduction in biocidal activity is mostly attributed to the factors such as chemical degradation of biocide, fast dissipation of biocide from application site due to washing out and/or volatilization. The life of any product where biocide is applied, will be more if these biocides are retained in the product/application site for longer period. This extended duration of biocidal activity can be achieved by incorporating biocide in Controlled Release (CR) form. Microcapsule is one of the best controlled release form, wherein an active agent (core material) is surrounded by a polymer film. This is achieved by a process called microencapsulation. Different techniques of microencapsulation are known which include phase separation, interfacial polymerization and mechanical methods such as spray drying. Numbers of reviews on microencapsulation techniques have appeared in literature. (i) Arshady R. (Editor), Microspheres, Microcapsules and Liposomes, Vol. 1 and 2, 1999 and Vol. 6, 2003 Citus Book, UK (ii) Benita S. (Editor), Microencapsulation-Methods and Industrial Applications, 1996, Marcel Dekker Inc., New York; (iii) Madan P. L., Asian J. Pharm. Sci., 9, 1979, p1; (iv) Thies C. In: Encyclopedia of Polymer Science and Engineering, Vol. 9, Wiley & Sons, New York, 1987, p 724 and (v) Porte H. and Couarrze G., In: Hand book of Powder Technology, 9 (Powder Technology and Pharm. Processes) 1994, p 513). U.S. patents disclosing various microencapsulation methods have been consolidated (Gutcho M. ‘Microcapsules and Microencapsulation Techniques’, New York, Noyes Data, 1976).

Controlled Release Biocides:

Controlled Release (CR) biocides has been the subject of interest to many researchers. CR concept and work was initiated first time on antifouling paints using chloroprene polymer by N. F. Cardarelli (Cardarelli N. F., Chapter 3 In Controlled Release Technologies: Methods, Theory & Applications, Ed. Kydonieus A. F., 1980, CRC Press Inc., USA). Antifouling marine paint composition containing gelation microcapsules of water-immiscible biocide has been described in U.S. Pat. No. 4,253,877. Yet another U.S. patent (U.S. Pat No. 5,378,413) describes preparation of gelatin microcapsules containing fouling reducing agents and their use in paint system. Another report describes extended control of marine fouling using formulation of microencapsulated organometallic biocide and vinyl rosin paint. (Porter R. and Miale J. B., Appl. Biochem. and Biotech., 9 (1984), p 439-445 (CA 102:162052)).

Apart from CR antifouling formulations there have been very few reports on CR of other biocides. Biocide namely 4,5-dichloro-n-octyl-3-isothiazolinone (DCOI) can be encapsulated in a variety of polysiloxane matrices using sol-gel chemistry (Ghosh T. and Nungesser E. N., Proc. Int. Symp. Control. Rel. Bioact. Mater., 25 (1998), p 324). The skin sensitization potential of active agent (3-isothiazolone) in loci such as water-based marine antifouling paint of decorative is reduced by encapsulating the active agent in polyurea (EP 679333 (1995), (CA 123: 332738). The fungicide tebuconazole and chlorothalonil were successfully incorporated into polyvinylpyridine (PVPy) and polyvinylpyridine-co-styrene nano particles (Liu Y. et. al., J. Appl. Poly. Sci, 79 (2001), p 458-465).

U.S. patent (U.S. Pat. No. 4,915,947) describes preparation of microencapsulated phytotoxic fungicides using crosslinked polyurea or polyamide to provide an effective agent for direct foliar application to control fungal diseases on crops. Urea -formaldehyde (UF) and/or melamine formaldehyde (MF) resins have been used to prepare microcapsules of fungicide namely 3-Iodo-2-propynylbutyl carbamate. These microcapsules when incorporated into exterior latex paint and applied onto rubberwood panels on exposing to the environment showed longer protection from discoloration. (Ibrahim W. A. et. al., Pertanika 12 (1989) p 409-412 (CA 114:25832)). The acrylic latex exterior paint containing microcapsules of fungicides 2,3,5,6-tetrachloro-4-methylsulfonylpryidine and tetrachloroisophthaonitrile have been reported to show good mildew protection (Noren G. K. et. al., J. Coatings Tech. 58 (1986), p 31-39 (CA 104: 188225)). Another patent describes encapsulation of biocide using MF resin and their use in coating material like plaster having silicate, mineral or polymer resin binder or a primer based on a silicate or polymer resin binder (Patent WO 2004000953). The patent describes preparation of microcapsules containing Zinc Pyrithione using MF resin but does not specify the size of the microcapsules obtained.

Japanese patent (No. JP 2003104802) describes antibacterial aqueous dispersion compositions containing microencapsulated dithioles or 2,2-dibromo-3-nitrilopropionamide and other microbiocides like Zinc Pyrithione. The patent does not describe preparation of microcapsules of Zinc Pyrithione but mentions that Zinc Pyrithione is added to composition containing microcapsules of other compounds as mentioned above.

Polyurethane Microcapsules:

Preparation of polyurethane microcapsules containing variety of active agents using aqueous continuous phase is known in the literature. (i) Fuyuma H., shinjo G. and Tsuji K., J. Pesticide Sci., 9, 1984, p 511 (ii) Choi K. y., Min K. S., Park I. H., Kim K. S. and Chang T., Polymer (Korea), 14, 1990, p 392 (iii) Ohtsuti T., Polymer, 32, 1991, p 2395 (iv) JP 04, 76016 (v) Reddy P. V. S, Mahesh G. N. Ramesh S., Sivakumar P. A. and Radhakrishnan G., Macromolecular Reports, A32 (suppls. 5 & 6), 1995, p 789 (v) JP 05, 194, 128 (CA 119:154041) (vi) JP 05, 201,814 (CA 119: 197763) (vii) Kim I. H, et. al., Polymer (Korea), 26, 2002, p 400-409 (CA 137:155715) (viii) Hong k. and Park S., Reactive & Functional Polymers, 42, 1999, p 193-200 (ix) Frere Y. et al., Eur. Poly. J., 34, 1998, p 193-199. Methods described in these reports involve use of aqueous phase and thus are only useful to encapsulate water insoluble active agents. Also the encapsulating wall material formed due to presence of water may contain polyurea moieties along with polyurethane.

U.S. Pat. No. 5,603,986 discloses preparation of polyurethane microcapsules containing coloring agents, catalysts and UV absorbents using non-aqueous continuous phase wherein block copolymer based on polydimethylsiloxane and polyalkylene oxide is used as surfactant. Applicants in their earlier invention (U.S. Pat. No. 5,962,003) have described preparation of polyurethane microcapsules containing water-soluble pesticide monocrotophos in non-aqueous medium. Block copolymer consisting of one block of diene or olefin polymer and another block of poly (alkylene oxide) was synthesized and used as surfactant.

Biocides play an important role in variety of applications. However reduction in biocidal activity due to factors such as chemical degradation of biocide and/or fast dissipation of biocide from the application site due to reasons like washing out with water, is a problem which leads to decrease in period of effectiveness of biocide.

In the prior art there neither exists any product like microcapsules of Irgarol and Zinc Pyrithione, in particular with specified particle size of 1-100 microns This is the first time that the microencapsules of Irgarol and Zinc Pyrithione have been prepared, for being used in paint.

Thus to satisfy the need to prolong the life of biocide, present invention provides polyurethane microcapsules of biocides like Irgarol and Zinc Pyrithione which are effective algaecide and fungicide respectively.

### OBJECTIVES OF THE INVENTION

The main object of the present invention is to provide microcapsules containing biocides, fungicide and algaecide in particular, using polyurethane as capsule wall material.

Another object of the present invention is to prepare polyurethane microcapsules of Zinc Pyrithione and Irgarol having particle size distribution in the range of 1-100 microns and preferably between 2-50 microns.

### SUMMARY OF THE INVENTION

Accordingly the present invention provides polyurethane microcapsules of biocide selected from Zinc Pyrithione and Irgarol having particle size in the range of 1-100 microns.

In an another embodiment of the present invention the encapsulating polymer used is polyurethane.

In yet another embodiment the ratio of biocide to polymer used is in the range of 0.3 to 3.0.

In yet another embodiment the biocide used is selected from Zinc Pyrithione and Irgarol.

In yet another embodiment the particle size of microcapsules obtained is preferably in the range of 1-50 microns.

In yet another embodiment the polyurethane microcapsules of biocide is useful for preparing coating materials such as interior and exterior paints, which consisting of an active agent biocide and polyurethane as encapsulating polymer.

Further the present invention provides a process for the preparation of polyurethane microcapsules of biocide selected from Zinc Pyrithione and Irgarol having particle size in the range of 1-100 microns, which comprises dispersing a biocide in an aliphatic hydrocarbon medium containing nonionic polymeric surfactant having hydrophilic and hydrophobic repeating units, diol or polyol having molecular weight in the range of 200-2000, adding a monomer, a catalyst and optionally a crosslinker to the above said dispersion under agitation, at a speed of 800-1000 rpm, and further, adding fumed silica and an isocyanate drop wise to the above said dispersion mixture, under agitation, for a period of 3-5 hours, at a temperature in the range of 40-50° C., and further for a second time, for a period of 12-15 hrs, at a temperature of 20-27° C., filtering and washing the resultant product with lower aliphatic hydrocarbon, followed by drying it under vacuum at a temperature of 20-35° C. to obtain the desired controlled particle size polyurethane microcapsules of biocide.

In an another embodiment the non polar aliphatic hydrocarbon solvent used is selected from the group consisting of hexane, octane, decane, isoctane, dodecane, hexadecane, superior kerosene, paraffin oil, white mineral oil and molax raffinate.

In yet another embodiment the ratio of hydrocarbon solvent to the total weight of polymer forming monomers and an active agent used is in the range of 1 to 10.

In yet another embodiment the ratio of hydrocarbon solvent to the total weight of polymer forming monomers and an active agent used is preferably in the range of 1.5 to 6.

In yet another embodiment the one of the polymer forming monomer used is diol selected from the group consisting of ethylene glycol, diethylene glycol, 1-4,butane diol and Poly(tetramethylene glycol).

In yet another embodiment the catalyst used is selected from amino and organometallic compound.

In yet another embodiment the catalyst used is an amino compound selected from the group consisting of N,N-dimethyl cyclohexylamine, N,N-dimethylcetylamine and diamino-bicyclooctane.

In yet another embodiment the catalyst used is an organometallic compound selected from stannous octoate and dibutyltin dilaurate.

In yet another embodiment the amount of catalyst used is in the range of 0.02 to 0.09 wt % based on total weight of polymer forming monomers.

In yet another embodiment the crosslinker used is selected from the group consisting of trimethylol propane, glycerol and hexane triols.

In yet another embodiment the amount of crosslinker used is 5 to 50 wt % based on diol.

In yet another embodiment the isocyanate monomer used is selected from the group consisting of toluene diisocyanate, methylene diisocyanate, isophorone diisocyanate, cyclohexane-1,4-diisocyanate, hexamethylene diisocyanate, m-tetramethyloxylene diisocyanate, 2,2,4- and 2,4,4-trimethyl hexamethylene diisocyanate and 2,5-norbornane diisocyanate.

In yet another embodiment the rate of adding icocyanate to the dispersion mixture used is in the range of 5 to 1.0 g per minute.

In yet another embodiment the equivalent ratio of isocyanate to diol used is in the range of 1.1 to 1.2.

In yet another embodiment the amount of fumed silica used is in the range of 0.2-0.9 wt % based on total weight of polymer forming monomers and biocide.

In yet another embodiment the temperature used in the reaction between isocyanate and diols or polyols is in the range of 25 to 60° C.

In yet another embodiment the nonionic polymeric surfactant used is in the range of selected from the group consisting of Hypermer 2296, Uniqema (HLB4.9), Hypermer A 60, Uniqema (HLB 6) and poly(lauryl methacrylate)-g-poly(ethylene oxide).

In yet another embodiment the concentration of non-polymeric surfactant used is in the range of 2-10 wt % based on total weight of polymer forming monomers and biocide.

In yet another embodiment the amount of surfactant used is preferably in the range of 3 to 9 wt % based on total weight of polymer forming monomers and biocide.

In yet another embodiment the quantity of biocide that is encapsulated is 25-70 wt. % of the total microcapsule weight.

### DETAIL DESCRIPTION OF THE INVENTION

The invention provides polyurethane microcapsules containing biocide, useful for preparing coating materials such as interior and exterior paints. Microcapsules described in this invention are prepared by dispersing biocide in an aliphatic hydrocarbon medium containing nonionic polymeric stabilizer having hydrophilic and hydrophobic repeating units, diol or polyol having molecular weight 200-2000, crosslinker and a catalyst selected from amino or organometallic compounds; adding an isocyanate drop wise to this dispersion; agitating the mixture at 800-1000 rotations per minute for the first 3-5 hours at 40-50° C. and then at 12-15 hours at 20-27° C.; to permit the formation of polyurethane microcapsules; filtering and washing the microcapsules with lower aliphatic hydrocarbon and drying the microcapsules under vacuum at temperature between 20-35° C.

In a feature the particle size can be controlled within more narrower limits by appropriate choice of the stabilizer, its concentration and agitator speed. This invention is further illustrated by the following examples which should not be construed to limit the scope of the invention.

### EXAMPLE 1

1 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 3 g is dispersed in this surfactant solution followed by addition of 0.5 g DBTDL catalyst solution (1.0 w/w % solution in paraffin oil) and 2 g of ethylene glycol and the mixture is agitated at 1000 rotations per minute using turbine type stirrer. To this mixture 0.1 g of fumed silica is added. After 15-30 minutes, 6.74 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours. The yield of the product is 11.3 g. Microcapsules have particle size range of 1-45 microns of which a majority of particles are 20-25 microns.

### EXAMPLE 2

1.28 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 14 g is dispersed in this surfactant solution followed by addition of 0.5 g DBTDL catalyst solution (1.0 w/w % solution in paraffin oil) and 2 g of ethylene glycol and the mixture is agitated at 1000 rotations per minute using turbine type stirrer. To this mixture 0.1 g of fumed silica is added. After 15-30 mintues, 6.74 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours. The yield of the product is 21.1 g. Microcapsules have particle size range of 5-50 microns of which a majority of particles are 10-20 microns.

### EXAMPLE 3

1.3 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 14 g is dispersed in this surfactant solution followed by addition of 0.5 g DBTDL catalyst solution (1.0 wt % solution in paraffin oil) and 2 g of ethylene glycol containing 0.2 g of trimethylol propane and the mixture is agitated at 1000 rotations per minute using turbine type stirrer. To this mixture 0.1 g of fumed silica is added. After 15-30 mintues, 6.99 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 22.4 g. Microcapsules have particle size range of 5-50 microns of which a majority of particles are 10-20 microns.

### EXAMPLE 4

1.3 g of polymeric surfactant Hypermer 2296 was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 14 g is dispersed in this surfactant solution followed by addition of 0.5 g DBTDL catalyst solution (1.0 wt % solution in paraffin oil) and 2 g of ethylene glycol containing 0.4 g of trimethylol propane and the mixture is agitated at 1000 rotations per minute using turbine type stirrer. To this mixture 0.1 g of fumed silica is added. After 15-30 minutes, 7.46 g of toluene diisocyanate is added drop wise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 23.0 g. Microcapsules have particle size range of 5-45 microns of which a majority of particles are 5-20 microns.

### EXAMPLE 5

2.9 g of polymeric surfactant Hypermer A60 was dissolved in 10 g of paraffin oil by heating the mixture at 65° C. In a jacketed reaction kettle having volume of 250 mL. 90 g of paraffin oil is taken followed by addition of above said surfactant solution at 25-27° C. Biocide namely Zinc Pyrithione 28 g is dispersed in this surfactant solution followed by addition of 1.0 g DBTDL catalyst solution (1.0 wt % solution in paraffin oil) and 4 g of ethylene glycol and the mixture is agitated at 1000 rotations per minute using turbine type stirrer. To this mixture 0.2 g of fumed silica is added. After 15-30 mintues, 13.5 g of toluene diisocyanate is added dropwise over a period of 20 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours.

The yield of the product is 44.9 g. Microcapsules have particle size range of 2-40 microns of which a majority of particles are 5-15 microns.

### EXAMPLE 6

0.82 g of polymeric surfactant Uniqema was dissolved in 5 g of paraffin oil by heating the mixture at 65° C. In a jacketed reaction kettle having volume of 250 mL. 45 g of paraffin oil is taken followed by addition of above said surfactant solution at 25-27° C. Biocide namely Zinc Pyrithione 7.3 g is dispersed in this surfactant solution followed by addition of 0.5 g DBTDL catalyst solution (1.0 wt % solution in paraffin oil) and 3 g of 2-ethyl 1,3 hexane glycol and the mixture is agitated at 1000 rotations per minute using turbine type stirrer. To this mixture 0.1 g of fumed silica is added. After 15-30 mintues, 4.28 g of toluene diisocyanate is added dropwise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours. The yield of the product is 13.3 g. Microcapsules have particle size range of 5-40 microns of which a majority of particles are 10-25 microns.

### EXAMPLE 7

2.98 g of polymeric surfactant Uniqema (HLB 6) was dissolved in 10 g of paraffin oil by heating the mixture at 65° C. In a jacketed reaction kettle having volume of 250 mL. 90 g of paraffin oil is taken followed by addition of above said surfactant solution at 25-27° C.

Biocide namely Irgarol 17.5 g is dispersed in this surfactant solution followed by addition of 1.0 g DBTDL catalyst solution (1.0 wt % solution in paraffin oil) and 4 g of ethylene glycol and the mixture is agitated at 1000 rotations per minute using turbine type stirrer. To this mixture 0.1 g of fumed silica is added. After 15-30 minutes, 13.5 g of toluene diisocyanate is added drop-wise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours. The yield of the product is 34.7 g. Microcapsules have particle size range of 2-50 microns of which a majority of particles are 5-20 microns.

### EXAMPLE-8

Polymeric surfactant namely poly(lauryl methacrylate)-g-poly(ethylene oxide) (number average molecular weight 34170, weight average molecular weight 96560) is synthesized as similar to procedure described in the literature (Palaskar D. V. et al., Proc. of International Seminar on Frontiers of Polymer Science and Engineering, Macro 2002, held at Khargpur, India, Dec. 9-11, 2002). 1.17 g of this surfactant was dissolved in 5 g of paraffin oil by heating the mixture at 60° C. In a jacketed reaction kettle having volume of 250 mL. 45 g of paraffin oil is taken followed by addition of above said surfactant solution at 25-27° C. Biocide namely Irgarol 14.0 g is dispersed in this surfactant solution followed by addition of 0.5 g DBTDL catalyst solution (1.0 wt % solution in paraffin oil) and 2 g of ethylene glycol and the mixture is agitated at 1000 rotations per minute using turbine type stirrer. To this mixture 0.1 g of fumed silica is added. After 15-30 minutes, 6.85 g of toluene diisocyanate is added drop wise over a period of 10 minutes. The temperature of reaction mixture is then raised to 40° C. After 4 hours the temperature of reaction mixture is brought to 25-27° C. and agitation speed is reduced to 500 rotations per minute. Thereafter by stirring the mixture for further 15 hours 25 mL of pet ether is added and stirred further for 10 minutes. The polyurethane microcapsules thus formed are isolated by centrifuging and washing with pet ether 3-4 times, filtering and drying under vacuum at 25° C. for 3-4 hours. The yield of the product is 21.2 g. Microcapsules have particle size range of 2-60 microns of which a majority of particles are 5-20 microns.

### ADVANTAGES

Biocides play an important role in variety of applications. However, reduction in biocidal activity due to factors such as chemical degradation of biocide and/or fast dissipation of biocide from the application site due to reasons like washing out with water is a problem which leads to decrease in period of effectiveness of biocide. Microencapsulated biocide will prolong the life of biocide and being encapsulated in polymer will result in less environmental pollution which unencapsulated biocide may cause.

1. Polyurethane microcapsules of biocide selected from Zinc Pyrithione and Irgarol having particle size in the range of 1-100 microns.

2. The polyurethane microcapsules of biocide as claimed in claim 1, wherein the encapsulating polymer used is polyurethane.

3. The polyurethane microcapsules of biocide as claimed in claim 1, wherein the ratio of biocide to polymer used is in the range of 0.3 to 3.0.

4. The polyurethane microcapsules of biocide as claimed in claim 1, wherein the biocide used is selected from Zinc Pyrithione and Irgarol.

5. The polyurethane microcapsules of biocide as claimed in claim 1, wherein the particle size of microcapsules obtained is preferably in the range of 1-50 microns.

6. The polyurethane microcapsules of biocide as claimed in claim 1, is useful for preparing coating materials such as interior and exterior paints, which consisting of an active agent biocide and polyurethane as encapsulating polymer.

7. A process for the preparation of polyurethane microcapsules of biocide selected from Zinc Pyrithione and Irgarol having particle size in the range of 1-100 microns, which comprises dispersing a biocide in an aliphatic hydrocarbon medium containing nonionic polymeric surfactant having hydrophilic and hydrophobic repeating units, diol or polyol having molecular weight in the range of 200-2000, adding a monomer, a catalyst and optionally a crosslinker to the above said dispersion under agitation, at a speed of 800-1000 rpm, and further, adding fumed silica and an isocyanate drop wise to the above said dispersion mixture, under agitation, for a period of 3-5 hours, at a temperature in the range of 40-50° C., and further for a second time, for a period of 12-15 hrs, at a temperature of 20-27° C., filtering and washing the resultant product with lower aliphatic hydrocarbon, followed by drying it under vacuum at a temperature of 20-35° C. to obtain the desired controlled particle size polyurethane microcapsules of biocide.

8. A process as claimed in claim 7, wherein the non polar aliphatic hydrocarbon solvent used is selected from the group consisting of hexane, octane, decane, isoctane, dodecane, hexadecane, superior kerosene, paraffin oil, white mineral oil and molax raffinate.

9. A process as claimed in claim 7, wherein the ratio of hydrocarbon solvent to the total weight of polymer forming monomers and an active agent used is in the range of 1 to 10.

10. A process as claimed in claim 7, wherein the ratio of hydrocarbon solvent to the total weight of polymer forming monomers and an active agent used is preferably in the range of 1.5 to 6.

11. A process as claimed in claim 7, wherein one of the polymer forming monomer used is diol selected from the group consisting of ethylene glycol, diethylene glycol, 1-4,butane diol and Poly(tetramethylene glycol).

12. A process as claimed in claim 7, wherein the catalyst used is selected from amino and organometallic compound.

13. A process as claimed in claim 7, wherein the catalyst used is an amino compound selected from the group consisting of N,N-dimethyl cyclohexylamine, N,N-dimethylcetylamine and diamino-bicyclooctane.

14. A process as claimed in claim 7, wherein the catalyst used is an organometallic compound selected from stannous octoate and dibutyltin dilaurate.

15. A process as claimed in claim 7, wherein the amount of catalyst used is in the range of 0.02 to 0.09 wt % based on total weight of polymer forming monomers.

16. A process as claimed in claim 7, wherein the crosslinker used is selected from the group consisting of trimethylol propane, glycerol and hexane triols.

17. A process as claimed in claim 7, wherein the amount of crosslinker used is 5 to 50 wt % based on diol.

18. A process as claimed in claim 7, wherein isocyanate monomer used is selected from the group consisting of toluene diisocyanate, methylene diisocyanate, isophorone diisocyanate, cyclohexane-1,4-diisocyanate, hexamethylene diisocyanate, m-tetramethyloxylene diisocyanate, 2,2,4- and 2,4,4-trimethyl hexamethylene diisocyanate and 2,5-norbornane diisocyanate.

19. A process as claimed in claim 7, wherein the rate of adding icocyanate to the dispersion mixture used is in the range of 5 to 1.0 g per minute.

20. A process as claimed in claim 7, wherein the equivalent ratio of isocyanate to diol used is in the range of 1.1 to 1.2.

21. A process as claimed in claim 7, wherein the amount of fumed silica used is in the range of 0.2-0.9 wt % based on total weight of polymer forming monomers and biocide.

22. A process as in claim 7, wherein the temperature used in the reaction between isocyanate and diols or polyols is in the range of 25 to 60° C.

23. A process as claimed in claim 7, wherein the nonionic polymeric surfactant used is in the range of selected from the group consisting of Hypermer 2296, Uniqema (HLB4.9), Hypermer A60, Uniqema (HLB 6) and poly(lauryl methacrylate)-g-poly(ethylene oxide).

24. A process as claimed in claim 7, wherein the concentration of non-polymeric surfactant used is in the range of 2-10 wt % based on total weight of polymer forming monomers and biocide.

25. A process as claimed in claim 7, wherein the amount of surfactant used is preferably in the range of 3 to 9 wt % based on total weight of polymer forming monomers and biocide.

26. A process as claimed in claim 7, wherein the quantity of biocide that is encapsulated is 25-70 wt. % of the total microcapsule weight.
